(19) United States
(12) Patent Application Publication
(10) Pub. No.: US 2025/0255501 A1
(43) Pub. Date: Aug. 14, 2025

(54) BLOOD PRESSURE MEASURING DEVICE

(71) Applicant: OMRON HEALTHCARE Co., Ltd., Kyoto (JP)

(72) Inventors: Yoshihide TOKKO, Kyoto (JP); Masaki HARADA, Kyoto (JP); Takashi ONO, Kyoto (JP); Yoshihiko SANO, Kyoto (JP); Yuki TAKANO, Kyoto (JP)

(21) Appl. No.: 19/058,735

(22) Filed: Feb. 20, 2025

Related U.S. Application Data
(63) Continuation of application No. PCT/JP2023/031133, filed on Aug. 29, 2023.

(30) Foreign Application Priority Data
Dec. 22, 2022 (JP) 2022-205664

(51) Int. Cl. A61B 5/022; A61B 5/00

(52) U.S. Cl. CPC A61B 5/02233; A61B 5/681

(57) ABSTRACT

A blood pressure measurement device includes a device body, a curler fixed to the device body, a fluid bag fixed to an inner surface of the curler and inflated by a fluid, and band provided integrally with an outer surface of the curler or the outer surface of the curler and an outer surface of the fluid bag and including a hook-and-loop fastener on an outer surface of the band.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/031133, filed Aug. 29, 2023, which application claims priority to Japanese Patent Application No. 2022-205664, filed Dec. 22, 2022, which applications are incorporated herein by reference in their entireties.

### TECHNICAL FIELD

The present invention relates to a blood pressure measurement device.

### BACKGROUND ART

In recent years, blood pressure measurement devices used for measuring a blood pressure are used as means to check health status at home, as well as in medical facilities. A blood pressure measurement device detects vibration of the artery wall to measure blood pressure by, for example, inflating and contracting a cuff wound around the upper arm or the wrist of a living body and detecting the pressure in the cuff by using a pressure sensor.

In addition, as the blood pressure measurement device, as disclosed in JP 2006-174860 A, there is known a wearable blood pressure monitor that is attached to a wrist. In such a wearable blood pressure monitor, an air bag and a curler are joined to each other, but the curler and a band are not joined to each other. Thus, a positional deviation may be generated between the curler and the band. When the positional deviation is generated, a portion in which the air bag and the curler cannot be suppressed by the band is generated, which may cause a decrease in measurement accuracy, deterioration of the air bag, or the like. In addition, an insert material is inserted in order to reinforce the band, but this increases a manufacturing cost of the band.

In addition, as disclosed in JP 2020-65651 A, there is also known a general wrist-type blood pressure measurement device in which the curler and the air bag are disposed in a cloth bag including an inner cloth and an outer cloth in order to prevent the positional deviation.

### CITATION LIST

### Patent Literature

Patent Document 1: JP 2006-174860 A
Patent Document 2: JP 2020-65651 A

### SUMMARY OF INVENTION

### Technical Problem

In the blood pressure measurement device in which the curler and the air bag are disposed in the cloth bag, it is necessary to use an easily stretchable material for the inner cloth so as not to hinder a bulge of the air bag. Thus, in a case of the blood pressure measurement device attached to the wrist, the blood pressure measurement device has a shape with a small radius of curvature when the blood pressure measurement device is attached, and a large sag or a wrinkle is generated in the inner cloth, which leads to deterioration in appearance.

Thus, an object of the present invention is to provide a blood pressure measurement device which can prevent positional deviations between a band and a curler and between the band and a fluid bag and has a good appearance and high accuracy.

### Solution to Problem

According to one aspect, there is provided a blood pressure measurement device including a device body, a curler fixed to the device body, a fluid bag fixed to an inner surface of the curler and inflated by a fluid, and a band provided integrally with an outer surface of the curler or the outer surface of the curler and an outer surface of the fluid bag and including a hook-and-loop fastener on an outer surface of the band.

According to this aspect, the band is formed integrally with the outer surface of the curler, or the band is formed integrally with the outer surfaces of the curler and the fluid bag, and thus generation of positional deviations between the band that fastens the fluid bag toward a living body and the curler and/or between the band and the fluid bag can be prevented. Thus, the blood pressure measurement device can stably press the fluid bag against the living body, and thus the accuracy of the blood pressure measurement is stabilized and the deterioration of the fluid bag can be prevented. In addition, the band is provided on the outer circumferential surface of a curler 51, and thus the generation of the sag or the wrinkle can be prevented, so that the blood pressure measurement device can obtain a high appearance.

There is provided the blood pressure measurement device according to the above-described aspect, in which the curler is formed longer than a length of the fluid bag in a longitudinal direction and is formed to be elastically deformable to follow a shape of the living body of an attachment site in a circumferential direction, and the band is provided on an outer circumferential surface of the curler and extends from a first end of the curler.

According to this aspect, the curler is elastically deformed to follow the shape of the living body and the band can fix the curler by a pair of the hook-and-loop fasteners, and thus the fluid bag can be pressed against the living body.

There is provided the blood pressure measurement device according to the above-described aspect, in which the curler is made of a low-hardness material and includes a fixed portion fixed to the device body and into which a high-hardness material having a hardness higher than a hardness of the low-hardness material is inserted, and the band is made of a material that is less likely to stretch than the curler.

According to this aspect, even when the curler is made of a material having a relatively low hardness, when the fluid bag is pressed toward the living body by the band and fastened by the hook-and-loop fastener, the curler can be bent and deformed to follow the living body. In addition, an outer circumferential surface of the curler is covered with the band made of a material that is less likely to stretch than the curler. Accordingly, the curler can be suppressed from being deformed outward in a radial direction when the fluid bag is inflated. Thus, the curler is easily elastically deformed to follow the shape of the living body, can be suppressed from being deformed outward in the radial direction during a blood pressure measurement, and can efficiently press the fluid bag toward the living body.

In addition, the curler is formed by inserting the high-hardness material having hardness higher than that of the material forming the curler into the fixed portion to be fixed to the device body, and thus an intensity of the fixed portion to be attached to the device body can be improved.

There is provided the blood pressure measurement device according to the above-described aspect, in which the device body includes a loop portion that folds back the band.

According to this aspect, the band makes it easy to fasten the curler and can bring the fluid bag into close contact with the living body stably.

There is provided the blood pressure measurement device according to the above-described aspect, in which a first end of both ends of the curler overlaps a second end of the both ends when the blood pressure measurement device is attached to a living body having a shortest length in a circumferential direction of at least the living body to which attachment is expected.

According to this aspect, the first end of the both ends of the curler overlaps the second end, and thus the fluid bag can be brought into contact with the living body even when the curler is attached to any of the living bodies having different circumferences. That is, the blood pressure measurement device prevents the both ends of the curler from coming into contact with each other in the circumferential direction to generate a gap between the living body and the fluid bag, and the fluid bag can be brought into close contact with the living body.

There is provided the blood pressure measurement device according to the above-described aspect, in which a first end of the curler has a smaller radius of curvature than a second end overlapping the first end of the curler.

According to this aspect, the second end of the curler is located outward in the radial direction than the first end before fastened by the band, and thus the second end of the curler can be disposed on an outer side of the first end when fastened by the band. Thus, in the blood pressure measurement device, when the curler is fastened by the band, the end portions easily overlap each other and the curler is easily wound around the wrist.

There is provided the blood pressure measurement device according to the above-described aspect, further including a guide portion that guides the second end overlapping the first end of the curler to an outer side of the first end.

According to this aspect, the second end can be guided to the first end of the curler by the guide portion, and thus the end portions of the curler can be easily overlapped each other.

There is provided the blood pressure measurement device according to the above-described aspect, in which the guide portion is a protrusion provided at the first end or the second end of the curler and moves the second end to an outer side.

According to this aspect, the guide portion is formed by the protrusion, and thus the curler is easily guided from one of the first end and the second end to the other.

There is provided the blood pressure measurement device according to the above-described aspect, in which the guide portion is a sheet member provided on an inner surface of the band that covers an end portion of the fluid bag and extends from the curler.

According to this aspect, the fluid bag and the second end of the curler is covered with the sheet member, and thus the first end of the curler can be suppressed from interfering with the fluid bag and the second end of the curler in the circumferential direction.

There is provided the blood pressure measurement device according to the above-described aspect, in which the curler is formed of a fixed portion made of a low-hardness material and a high-hardness material inserted into the low-hardness material and having a hardness higher than a hardness of the low-hardness material is inserted, the fixed portion being fixed to the device body, and the outer surface of the fluid bag is integrally fixed to the fixed portion and the band.

According to this aspect, even when the curler is made of a material having a relatively low hardness, when the fluid bag is pressed toward the living body by the band and fastened by the hook-and-loop fastener, the curler can be bent and deformed to follow the living body. In addition, the fluid bag is configured such that the outer circumferential surface is covered with the band. Accordingly, the fluid bag can be suppressed from being deformed outward in the radial direction when the fluid bag is inflated. Thus, the fluid bag is easily deformed to follow the shape of the living body, and the fluid bag is suppressed from being deformed outward in the radial direction during a blood pressure measurement and can be efficiently pressed toward the living body.

In addition, the curler is formed by inserting the high-hardness material having hardness higher than that of the material forming the curler into the fixed portion to be fixed to the device body, and thus an intensity of the fixed portion to be attached to the device body can be improved.

There is provided the blood pressure measurement device according to the above-described aspect, further including a sensor provided on an inner surface of a first end portion of the curler and separated from the fluid bag in a longitudinal direction of the curler and detects information on a living body.

According to this aspect, the sensor for detecting the information on the living body is provided in the curler, and thus the sensor can be brought into contact with the living body, and the information of the living body other than the blood pressure can be detected.

### Advantageous Effects of Invention

According to the present invention, a blood pressure measurement device which can prevent positional deviations between a band and a curler and between the band and a fluid bag and has a good appearance and high accuracy can be provided.

### BRIEF DESCRIPTION OF DRAWINGS

FIG. 1 is a perspective view illustrating a configuration of a blood pressure measurement device according to an embodiment of the present invention.

FIG. 2 is a side view illustrating the configuration of the blood pressure measurement device.

FIG. 3 is a side view illustrating the configuration of the blood pressure measurement device in a state of being attached to the wrist.

FIG. 4 is a block diagram illustrating the configuration of the blood pressure measurement device.

FIG. 5 is a perspective view illustrating configurations of a cuff structure and a band used in the blood pressure measurement device.

FIG. 6 is a side view illustrating a configuration of a main part of a cuff structure according to another embodiment of the present invention.

FIG. 7 is a side view illustrating a configuration of a main part of a cuff structure according to another embodiment of the present invention.

FIG. 8 is a side view illustrating a configuration of a main part of a cuff structure according to another embodiment of the present invention.

FIG. 9 is a perspective view illustrating a configuration of a blood pressure measurement device according to another embodiment of the present invention.

FIG. 10 is a perspective view illustrating a configuration of a blood pressure measurement device according to another embodiment of the present invention.

FIG. 11 is a perspective view illustrating a configuration of a blood pressure measurement device according to another embodiment of the present invention.

### DESCRIPTION OF EMBODIMENTS

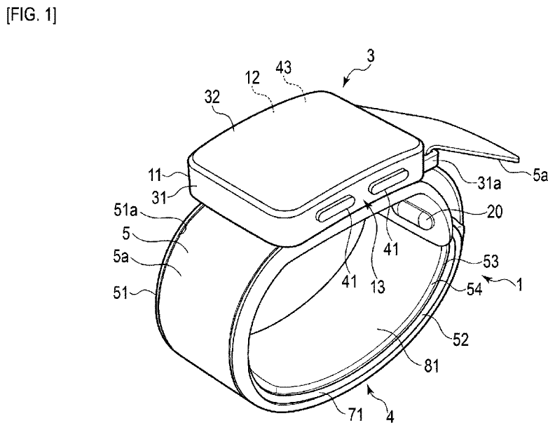

Hereinafter, an example of a blood pressure measurement device 1 according to an embodiment of the present invention will be described with reference to FIG. 1 to FIG. 5.

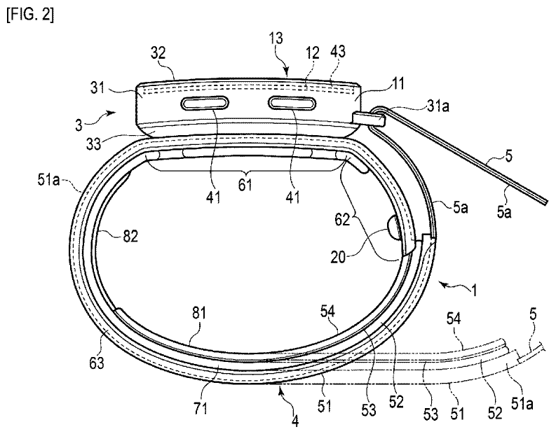

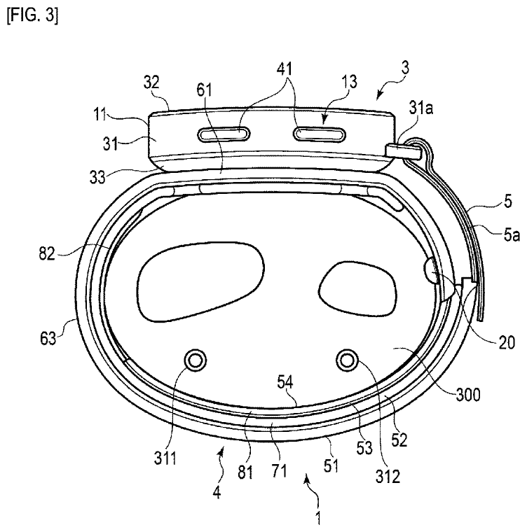

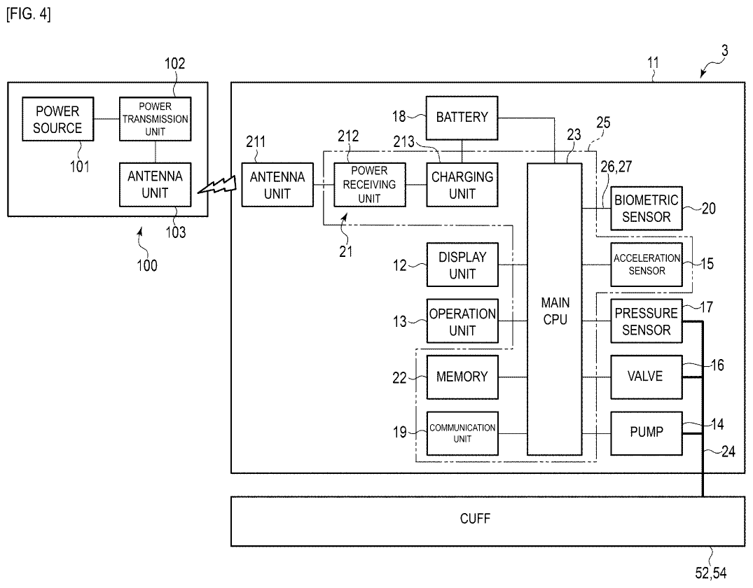

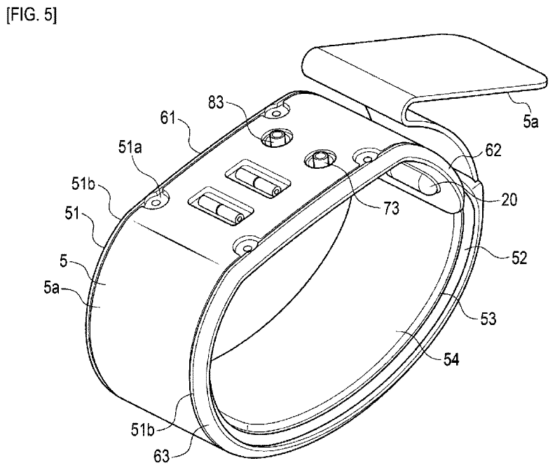

FIG. 1 is a perspective view illustrating a configuration of the blood pressure measurement device 1 according to the embodiment of the present invention, and FIG. 2 is a side view illustrating the configuration of the blood pressure measurement device 1. FIG. 3 is a side view illustrating the configuration of the blood pressure measurement device 1 in a state of being attached to a wrist 300. FIG. 4 is a block diagram illustrating a configuration of a device body 3 of the blood pressure measurement device 1. FIG. 5 is a perspective view illustrating configurations of a cuff structure 4 and a band 5 used in the blood pressure measurement device 1.

The blood pressure measurement device 1 is an electronic blood pressure measurement device attached to a living body. In an example of the present embodiment, the blood pressure measurement device 1 is a wearable device attached to the wrist 300 as illustrated in FIG. 3. The blood pressure measurement device 1 is, for example, an electronic blood pressure measurement device having an aspect of measuring a blood pressure from arteries 311 and 312 of the wrist 300.

As illustrated in FIGS. 1 to 3, the blood pressure measurement device 1 includes the device body 3, the cuff structure 4, and a band 5.

As illustrated in FIGS. 1 to 4, the device body 3 includes, for example, a housing 11, a display unit 12, an operation unit 13, a pump 14, an acceleration sensor 15, a valve 16, a pressure sensor 17, a battery 18, a communication unit 19, a charging circuit unit 21, a memory 22, and a processor 23.

The housing 11 is a case that accommodates components of the device body 3. The housing 11 accommodates, for example, the display unit 12, the operation unit 13, the pump 14, the acceleration sensor 15, the valve 16, the pressure sensor 17, the battery 18, the communication unit 19, a biometric sensor 20, the charging circuit unit 21, the memory 22, and the processor 23. The housing 11 accommodates, for example, a fluid circuit 24 and a substrate 25. Here, the fluid circuit 24 may include, for example, a pipe or a flow path plate that forms a flow path of a fluid supplied from the pump 14 to the cuff structure 4, a component such as a fluid resistor that controls a supply amount or a pressure of the fluid supplied to the cuff structure 4, and a check valve that controls a flow direction of the fluid. For example, the acceleration sensor 15, the communication unit 19, the circuit configuration of the charging circuit unit 21, the memory 22, and the processor 23 are mounted on the substrate 25.

The housing 11 includes, for example, an outer case 31, a windshield 32 that covers an upper opening of the outer case 31, and a back lid 33 that covers a lower portion of the outer case 31.

The outer case 31 is formed in, for example, a cylindrical shape, a rectangular cylindrical shape, or a polygonal cylindrical shape. In the present embodiment, the outer case 31 is formed in the rectangular cylindrical shape. The outer case 31 includes a loop portion 31a provided on one surface of an outer circumferential surface. The loop portion 31a is a rectangular annular member including an opening that is long in one direction and through which the band 5 can be inserted in order to allow the band 5 to pass the loop portion 31a and to fold back the band 5. The loop portion 31a is integrally formed on an outer surface of the outer case 31. The windshield 32 is a glass plate having the same shape as that of an outer circumferential edge of the outer case 31, that is, a rectangular shape in the present embodiment. The windshield 32 is not limited to the glass plate as long as the windshield 32 is made of a transparent or translucent material. The back lid 33 closes the bottom of the outer case 31. The housing 11 may have a configuration in which the back lid 33 is not included, and the bottom of the outer case 31 may be closed by a curler 51 (described later) of the cuff structure 4, the curler 51 being fixed to the housing 11.

The display unit 12 is disposed directly below the windshield 32. The display unit 12 is electrically connected to the processor 23. The display unit 12 is, for example, a liquid crystal display or an organic electroluminescence display. The display unit 12 displays various types of information including the date and time, measurement results of blood pressure values such as the systolic blood pressure and diastolic blood pressure, heart rate, a charging status and a remaining amount of the battery 18, and the like. For example, the display unit 12 is formed in the same shape as the windshield 32 in a plan view.

The operation unit 13 is configured to be capable of inputting a command from a user. The operation unit 13 includes, for example, a plurality of buttons 41 provided on the housing 11, a sensor that detects operation of each of the buttons 41, and a touch panel 43 provided on the display unit 12 or the windshield 32. When the operation unit 13 is operated by the user, the operation unit 13 converts a command into an electrical signal. The sensor and the touch panel 43 are electrically connected to the processor 23 to output electrical signals to the processor 23.

The pump 14 is, for example, a piezoelectric pump. The pump 14 compresses air as a fluid, for example, and supplies the compressed air to the pressing cuff 52 and the sensing cuff 54, which are described later, of the cuff structure 4 via the fluid circuit. The pump 14 is electrically connected to the processor 23.

The acceleration sensor 15 is, for example, a 3-axis acceleration sensor. The acceleration sensor 15, for example, measures acceleration and outputs an analog signal. The acceleration sensor 15 is, for example, connected to the processor 23 via an A/D conversion circuit.

The valve 16 is, for example, an on-off valve. The valve 16 opens and closes a fluid circuit connecting the pump 14 to the cuff structure 4 and/or a fluid circuit connecting the cuff structure 4 to the atmosphere. The valve 16 is electrically connected to the processor 23. For example, the valve 16 is opened and closed by control of the processor 23.

As a specific example, the valve 16 is a safety valve that releases air supplied to the pressing cuff 52 and the sensing cuff 54 (which are described later) of the cuff structure 4 to the atmosphere. The valve 16 is switched to a closed state by being controlled by the processor 23 when air is supplied to the pressing cuff 52 and the sensing cuff 54 during a blood pressure measurement. In addition, when the air in the pressing cuff 52 and the sensing cuff 54 is exhausted, the valve 16 is controlled by the processor 23 so as to be switched from the closed state to the open state. Further, the valve 16 may be formed such that the opening degree is adjustable. The valve 16 may be provided on the fluid circuit 24, or may be integrally provided inside s housing of the pump 14.

The pressure sensor 17 is provided, for example, in the fluid circuit 24. The pressure sensor 17 detects the pressures of the pressing cuff 52 and/or the sensing cuff 54. For example, the pressure sensor 17 detects the pressure of the sensing cuff 54. The pressure sensor 17 is electrically connected to the processor 23 via the A/D conversion circuit, converts detected pressure into an electrical signal, and outputs the electrical signal to the processor 23.

The battery 18 is, for example, a secondary battery such as a chargeable and dischargeable lithium ion battery. The battery 18 is electrically connected to the processor 23. The battery 18 supplies power to the processor 23. The battery 18 supplies power for driving to the respective configurations of the processor 23 and the display unit 12, the operation unit 13, the pump 14, the acceleration sensor 15, the valve 16, the pressure sensor 17, and the communication unit 19 via the processor 23.

The communication unit 19 can transmit and receive information to and from an external device wirelessly and/or by wire. The communication unit 19 is, for example, a wireless communication module conforming to a wireless communication standard. The communication unit 19 transmits information, such as information controlled by the processor 23 and measured blood pressure values and pulse, to an external device, or receives, for example, a program or recording medium for software update from an external device and transmits this to the control unit. In the present embodiment, the external device is, for example, an external terminal, such as a smartphone, a tablet terminal, a personal computer, and a smart watch.

In the present embodiment, the communication unit 19 and the external terminal may be directly connected, or may be connected over a network. The communication unit 19 and the external terminal may be connected via a mobile communication network, such as 4G and 5G, and a wireless communication line, such as Wimax and Wi-Fi (registered trademark). Further, the communication unit 19 and the external device may be connected by wireless communication means, such as Bluetooth Low Energy (BLE (registered trademark)), Near Field Communication (NFC), and infrared communication. Furthermore, in addition to the wireless communication module, the communication unit 19 may include a universal connector such as a micro Universal Serial Bus (USB) or a dedicated connector for the blood pressure measurement device 1, and may be directly connected to the external terminal by various cables such as a USB cable or connected to the external terminal via a wired communication line such as a Local Area Network (LAN) connection. Thus, the communication unit 19 may include a plurality of communication means, such as a wireless antenna and a micro-USB connector. The connector for wired communication may be a dedicated connector for the blood pressure measurement device 1.

The biometric sensor 20 is a sensor that is formed so as to be capable of detecting the information on a living body by coming into contact with or facing the wrist 300. The biometric sensor 20 converts the detected information on a living body into an electrical signal and outputs the electrical signal to the processor 23. The biometric sensor 20 may be, for example, a sensor that measures a physical quantity such as a heart rate or a body temperature, or may be a sensor that measures a chemical value such as a blood glucose level or a blood oxygen concentration. For example, the biometric sensor 20 is provided on the back lid 33 of the housing 11 and/or the curler 51 (described later) of the cuff structure 4. In the present embodiment, an example is illustrated in which the biometric sensor 20 is provided on the curler 51. The blood pressure measurement device 1 need not include the biometric sensor 20. The biometric sensor 20 is connected to the substrate 25 via, for example, a pogo pin 26 and a flexible printed circuit (FPC) 27. Here, the pogo pin is, for example, a spring probe or a contact probe.

The charging circuit unit 21 includes, for example, an antenna unit 211, a power receiving unit 212, and a charging unit 213. The charging circuit unit 21 charges the battery 18 by wireless power feeding. For example, the charging circuit unit 21 receives transmission power transmitted from the antenna unit 103 of a power transmission device 100 provided outside and charges the battery 18.

The antenna unit 211 receives the transmission power from the antenna unit of the power transmission device. The antenna unit 211 is, for example, a receiver coil as a power receiving resonance circuit. The antenna unit 211 supplies the received power to the power receiving unit 212. A power receiving surface of the antenna unit 211 is formed in a planar shape. The antenna unit 211 is disposed, for example, in the housing 11. As a specific example, the antenna unit 211 is provided in the housing 11 and adjacent to the display unit 12 on a side opposite to the windshield 32 of the display unit 12. The antenna unit 211 includes, for example, a resonance capacitor and constitutes the power receiving resonance circuit.

The power receiving unit 212 rectifies the power received by the antenna unit 211 and supplies the rectified power to the charging unit 213. As a specific example, the power receiving unit 212 rectifies the received power supplied from the antenna unit 211 and converts rectified received power from AC to DC. For example, the power receiving unit 212 includes a rectifying circuit and a control circuit, controls the operation of the rectifying circuit by the control circuit, and outputs rectified DC power to the charging unit 213.

The charging unit 213 supplies the power supplied from the power receiving unit 212 to the battery 18 as power for charging. For example, the charging unit 213 converts the power supplied from the power receiving unit 212 into a predetermined current value and a predetermined voltage value and supplies the converted power to the battery 18. Further, for example, the charging unit 213 may include a circuit that outputs the charge state of the battery 18 to the power receiving unit 212 and/or the processor 23.

The memory 22 includes, for example, a Random Access Memory (RAM) and a Read Only Memory (ROM). The memory 22 stores various data. For example, the memory 22 stores in advance, for example, programs/recording mediums and various program/recording medium data such as applications for controlling the entire blood pressure measurement device 1 and the pump 14, settings data for setting various functions of the blood pressure measurement device 1, and calculation data for calculating a blood pressure value and a pulse from the pressure measured by the pressure sensor 17 to be changeable.

The processor 23 controls the operation of the entire blood pressure measurement device 1 and the operations of the pump 14 and the valve 16 based on the programs or recording mediums stored in the memory 22 to perform a predetermined operation (function). The processor 23 executes the predetermined operation, analysis, processing, or the like according to the read program or recording medium. The processor 23 is an arithmetic device such as a CPU. The processor 23 may include, for example, a sub CPU in addition to a main CPU. In addition, the processor 23 displays statuses or results of the executed various operations, calculations, analyses, processes, and the like on the display unit 12 by the program/recording medium or the application.

The cuff structure 4 includes, for example, the curler 51, the pressing cuff 52, the back plate 53, and the sensing cuff 54. The cuff structure 4 is formed by layering the curler 51, the pressing cuff 52, the back plate 53, and the sensing cuff 54.

Hereinafter, specific examples of the cuff structure 4 will be described with reference to FIGS. 1 to 3 and 5. As illustrated in FIGS. 1 to 3 and 5, the cuff structure 4 includes the curler 51, the pressing cuff 52, the back plate 53, and the sensing cuff 54.

For example, a first end side of the curler 51 is fixed to the wrist side of the housing 11. The curler 51 is formed in a band-like shape that curves in a shape following along the circumferential direction of the wrist 300. The curler 51 is constituted by a resin material. The curler 51 is made of a material having a low hardness and having flexibility and shape retainability. Here, “flexibility” refers to deformation of the shape of the curler 51 in a radial direction at the time of application of an external force of the band 5 to the curler 51. “Shape retainability” refers to the ability of the curler 51 to maintain a pre-imparted shape when no external force is applied to the curler 51. That is, the curler 51 is made of a resin material having a hardness that does not undergo compressive deformation or substantially does not undergo compressive deformation but allows elastic deformation such as bending deformation in which the shape, in particular, a curvature of a curved portion changes. Thus, the curler 51 is formed to be elastically deformable so that the curler 51 is bent and deformed by the application of the external force and an inner space in which the wrist 300 is disposed becomes larger or smaller to follow the shape of the wrist to which the curler 51 is attached. For example, the curler 51 is made of a thermoplastic polyurethane resin (hereinafter referred to as TPU).

For example, a groove 51a to which the band 5 is disposed and fixed is formed on the outer circumferential surface of the curler 51. In other words, the curler 51 includes a pair of walls 51b along a longitudinal direction of the outer circumferential surface at a pair of edges along the longitudinal direction, and a space between the pair of walls 51b is the groove 51a to which the band 5 is disposed and fixed. For example, the groove 51a is formed from a first end portion to a second end portion of the curler 51 along the outer circumferential surface of the curler 51.

In addition, the curler 51 is formed such that a first side from a portion fixed to the housing 11 is longer than a second side. For example, both ends of the curler 51 are formed to have a length and a shape so as to be located at a first side portion of the wrist 300 between a palm side of a hand and a back side of the hand of the wrist 300 even when the curler 51 is attached to either the wrist 300 having the longest circumference or the wrist 300 having the shortest circumference among users to whom the curler 51 is expected to be attached. For example, the curler 51 is formed in a shape in which any one of the first end portion and the second end portion overlaps the other. For example, the curler 51 is inclined such that an end surface of an end portion located inward in the radial direction in a state where the both ends overlap each other faces the overlapped end portion side. In addition, the curler 51 may have a shape in which the entirety of the curler 51 curved in the longitudinal direction so as to follow the shape of the wrist 300 or part of the curler 51 is partially formed in a flat plate shape.

As a specific example, the curler 51 is, for example, fixed to the outer case 31 or the back lid 33 of the housing 11. The curler 51 includes a fixed portion 61 fixed to the back lid 33 and formed in a flat plate shape, a first curved portion 62 provided at a first end of the fixed portion 61 and curved with a predetermined radius of curvature, and a second curved portion 63 provided at a second end of the fixed portion 61 and curved with a predetermined radius of curvature.

A reinforcing material made of a high-intensity material such as polycarbonate (PC) is inserted into the fixed portion 61 by, for example, insert molding. The inserted reinforcing material is a material forming the curler 51, that is a material having a hardness higher than that of TPU in this embodiment. In addition, for example, a hole in which a fastening member such as a screw for fixing the curler 51 to the housing 11, connecting portions 73 and 83, a valve provided in the fluid circuit 24, and the like are disposed is formed in the fixed portion 61.

The first curved portion 62 is formed to have a length from the fixed portion 61 to the first side portion of the wrist 300. In addition, for example, the biometric sensor 20 is provided on the inner surface of the end portion side of the first curved portion 62. The second curved portion 63 is formed to have a length from the fixed portion 61 to the first side portion of the wrist 300 on which the end portion of the first curved portion 62 is disposed, beyond a second side portion of the wrist 300 and the palm side of the hand of the wrist 300.

For example, as indicated by a two dot chain line in FIG. 2, in a state before the external force is applied to the curler 51, the end portion of the second curved portion 63 may be located further outward in the radial direction than the end portion of the first curved portion 62 and separated in the circumferential direction, and the end portions of the first curved portion 62 and the second curved portion 63 may be separated from each other. As a specific example, the second curved portion 63 is formed such that the entire region or a part of the second curved portion 63 from the palm side of the hand to the end portion side is formed in a straight shape, the second curved portion 63 is formed in a curved shape having a single radius of curvature larger than the radius of curvature of the end portion side of the first curved portion 62 or in a curved shape having a plurality of different radii of curvature, or the second curved portion 63 is formed by a combination of the straight shape and the curved shape.

The curler 51 is formed so as to be capable of performing the overlap in which when the curler 51 is fastened by the band 5 toward the wrist 300 side, for example, one of the end portions of the first curved portion 62 and the end portion of the second curved portion 63 is located further outward in the radial direction than the other. As a specific example, as illustrated in FIGS. 2 and 3, the end portion of the second curved portion 63 is located further outward in the radial direction than the end portion of the first curved portion 62, and when the curler 51 is fastened by the band 5 toward the wrist 300 side the second curved portion 63 is formed so as to be capable of overlapping an outer surface side of the first curved portion 62. In other words, the end portion of the second curved portion 63 is formed to be capable of overlapping the end portion of the first curved portion 62. For example, in the first curved portion 62, the inner circumferential surface side is formed to be longer than the outer circumferential surface side, and thus the end surface is inclined with respect to the longitudinal direction of the first curved portion 62.

Note that, for example, the lengths of the first curved portion 62 and the second curved portion 63 may be configured such that the first curved portion 62 and the second curved portion 63 overlap each other in all the wrists 300 to which attachment is expected. In addition, for example, the first curved portion 62 and the second curved portion 63 may be configured such that the first curved portion 62 and the second curved portion 63 overlap each other when the blood pressure measurement device 1 is attached to a wrist 300 having a specific circumference, for example, from a wrist 300 having the median circumference to a wrist 300 having the shortest circumference among the wrists 300 to which the blood pressure measurement device 1 is expected to be attached.

In addition, a sensor attachment portion provided with the biometric sensor 20 is formed on an inner surface of the first curved portion 62 or the second curved portion 63 which is located inward in the radial direction when overlapping each other among the first curved portion 62 and the second curved portion 63. In the present embodiment, the sensor attachment portion is formed in the first curved portion 62, and the biometric sensor 20 is attached to the inner surface on a distal end side of the first curved portion 62. The sensor attachment portion is provided to be separated from the air bags 71 and 81 of the pressing cuff 52 and the sensing cuff 54, respectively, fixed to an inner circumferential surface of the curler 51 in the circumferential direction of the curler 51.

The lengths of the first curved portion 62 and the second curved portion 63 are lengths by which even in the wrist 300 having any circumference, the second curved portion 63 faces a site of the wrist 300 where the two arteries 311 and 312 are present, the second curved portion 63 is disposed on a part of the side portion of the wrist 300, and the end portion of the first curved portion 62 and the end portion of the second curved portion 63 are separated from each other. The two arteries 311 and 312 as used herein are the radial artery 311 and the ulnar artery 312.

The curler 51 has the largest curvature, for example, at a boundary (ridge portion) between the fixed portion 61 and the first curved portion 62 and at a boundary (ridge portion) between the fixed portion 61 and the second curved portion 63.

For example, the curler 51 is insert-molded in a rectangular flat plate shape, and then the band 5 is bonded to the groove 51a, and the curler 51 is formed in a predetermined curved shape using a molding die.

The pressing cuff 52 is fixed to the inner circumferential surface of the curler 51 by a double-sided tape, an adhesive, thermal welding, or the like. The pressing cuff 52 is provided at least in a region of the second curved portion 63 where the artery of the wrist 300 is present. As a specific example, the pressing cuff 52 is provided in a region of the curler 51 in the longitudinal direction from the fixed portion 61 side of the first curved portion 62 including the ridge portion between the fixed portion 61 and the first curved portion 62 to the end portion side of the second curved portion 63. The pressing cuff 52 extends along the inner surface of the curler 51.

The pressing cuff 52 is fluidly connected to the pump 14 through the fluid circuit 24. A first main surface of the pressing cuff 52 is fixed to the inner surface of the curler 51. The pressing cuff 52 is inflated to press the back side of the hand of the wrist 300 and press the back plate 53 and the sensing cuff 54 toward the wrist 300 side.

The pressing cuff 52 includes, for example, a single or a plurality of air bags 71, and a connecting portion 73 such as a nipple provided in the air bag 71 and connected to the fluid circuit 24. Here, the air bag 71 is a bag-like structure, and in the present embodiment, the blood pressure measurement device 1 is configured to use air with the pump 14, and thus the present embodiment will be described using the air bag. However, in a case where a fluid other than air is used, the bag-like structure may be any fluid bag that is inflated by the fluid. The air bag 71 is formed in a rectangular bag shape that is long in one direction.

The air bag 71 is formed in a bag shape by thermal welding or the like of a plurality of sheet members. For example, when the pressing cuff 52 is configured to include the plurality of air bags 71, the plurality of air bags 71 are laminated, integrally formed by welding or the like, and fluidly continuous with each other. The sheet member forming the air bag 71 is made of, for example, a thermoplastic elastomer. As the thermoplastic elastomer, for example, TPU is used.

The back plate 53 is fixed to the surface of the pressing cuff 52 on the wrist 300 side with a double-sided tape, an adhesive, or the like. The back plate 53 is formed of a resin material. The back plate 53 is formed in a rectangular plate shape that is long in one direction, for example. For example, the back plate 53 may be configured to be divided, that is, may be formed by arranging a plurality of rectangular small pieces in one direction. The back plate 53 has shape followability.

Here, “shape followability” refers to a function in which the back plate 53 can be deformed in such a manner as to follow the shape of a contacted portion of the wrist 300 to be disposed. This contacted portion of the wrist 300 refers to a region of the wrist 300 that comes into contact with the back plate 53. This contact includes both direct contact with the back plate 53 and indirect contact with the back plate 53 with the sensing cuff 54 interposed therebetween.

The sensing cuff 54 is fixed to the main surface of the back plate 53 on the wrist side. The sensing cuff 54 comes into direct contact with a region of the wrist 300 where the arteries 311 and 312 are present or indirect contact with the region with the cover or the like interposed therebetween. The sensing cuff 54 is formed in a rectangular shape that is long in one direction. The sensing cuff 54 may be configured to come into direct contact with a region of the wrist 300 where one of arteries 311 and 312 are present. The sensing cuff 54 is smaller than the pressing cuff 52 in the longitudinal direction. The sensing cuff 54 is the same as or smaller than the pressing cuff 52 in the lateral direction. The sensing cuff 54 is in the same shape as that of the back plate 53 or is smaller than that of the back plate 53, in the longitudinal direction and the width direction of the back plate 53. The sensing cuff 54 compresses the region on the palm side of the hand of the wrist where the artery is present by being inflated. The sensing cuff 54 is pressed by the inflated pressing cuff 52 toward the living body side with the back plate 53 interposed therebetween.

As a specific example, the sensing cuff 54 includes the air bag 81, a flow path body 82 fluidly connected to the air bag 81, and a connecting portion 83a provided to the flow path body 82. The air bag 81 and the flow path body 82 are formed in a bag shape by thermal welding or the like of a plurality of sheet members. The sheet member forming the air bag 81 and the flow path body 82 is made of, for example, a thermoplastic elastomer. As the thermoplastic elastomer, for example, TPU is used.

Here, the air bag 81 is a bag-like structure, and in the present embodiment, the blood pressure measurement device 1 is configured to use air with the pump 14, and thus the present embodiment will be described using the air bag. However, in a case where a fluid other than air is used, the bag-like structure may be any fluid bag that is inflated by the fluid. The air bag 81 is constituted in a rectangular shape that is long in one direction.

The flow path body 82 is, for example, integrally provided at a part of one edge of the air bag 81 in the longitudinal direction. The flow path body 82 is provided at the end portion of the air bag 81 near the device body 3. In addition, the flow path body 82 is formed in a shape which is long in one direction with a width smaller than the width of the air bag 81 in the lateral direction. The connecting portion 83 is integrally provided at a distal end of the flow path body 82. The flow path body 82 is connected to the fluid circuit 24 via the connecting portion 83 and forms a flow path between the fluid circuit 24 and the air bag 81. The connecting portion 83 is, for example, a nipple.

The band 5 is formed in a band-like shape. The band 5 includes, for example, a pair of hook-and-loop fasteners 5a in which a hook is formed on a first side and a loop is formed on a second side, and when the pair of hook-and-loop fasteners 5a engage with each other, the band 5 whose end portion side is inserted into the loop portion 31a is fixed.

The band 5 is formed integrally with the curler 51 or the pressing cuff 52, fastens the curler 51 or the pressing cuff 52 to the wrist 300, and brings the pressing cuff 52 and the sensing cuff 54 into close contact with the wrist 300. That is, the band 5 is formed so as to be capable of pressing the pressing cuff 52 and the sensing cuff 54 toward the wrist 300 by fastening the curler 51 so as to deform the curler 51 toward the wrist 300.

In the present embodiment, the band 5 is welded or attached to the groove 51a of the curler 51, and is formed integrally with the curler 51. The band 5 extends from one of the end portions which is located on the outer side when overlapping each other among the first curved portion 62 and the second curved portion 63 of the curler 51. In the present embodiment, the band 5 extends from the end portion of the second curved portion 63, and the loop portion 31a is provided on the outer surface of the outer case 31 on the side where the first curved portion 62 is provided. When the band 5 fastens the wrist 300 having the maximum circumference to which attachment is expected, the band 5 is inserted into the loop portion 31a provided in the outer case 31 and extends from the second curved portion 63 by a foldable length.

In the band 5, for example, a pair of hook-and-loop fasteners 5a is provided on a first main surface of a cloth material, and the groove 51a of the curler 51 is attached to a second main surface. The cloth material of the band 5 is made of a material that is less likely to stretch than the curler 51. The band 5 reinforces the curler 51 by attaching the cloth material to the curler 51. In other words, the band 5 is made of the cloth material that is less likely to stretch than the curler 51, and is attached to the curler 51, thereby suppressing the curler 51 from deforming to an outer side in the radial direction.

Next, an example of the power transmission device 100 that transmits power to the charging circuit unit 21 of the device body 3 will be described.

As illustrated in FIG. 4, the power transmission device 100 includes a power source 101, a power transmission unit 102, and an antenna unit 103. The power source 101 is, for example, an AC adapter or the like connected to a commercial power source or the like. The power source 101 converts AC power input from the commercial power source into DC power and supplies the DC power to the power transmission unit 102.

The power transmission unit 102 generates AC power as transmission power from the DC power supplied from the power source 101, and supplies the AC power to the antenna unit 103. For example, the power transmission unit 102 generates AC power having a frequency that is the same or substantially the same as a resonance frequency of a power transmission resonance circuit of the antenna unit 103.

The antenna unit 103 is, for example, a transmitter coil as the power transmission resonance circuit. A power transmission surface of the antenna unit 103 is formed in a planar shape. The antenna unit 103 transmits power to the antenna unit 211 of the device body 3. The antenna unit 103 includes, for example, a resonance capacitor and constitutes the power transmission resonance circuit.

According to the blood pressure measurement device 1 configured as described above, the band 5 is integrally provided on the outer circumferential surface of the curler 51, and thus a positional deviation between the band 5 and the curler 51, that is, a positional deviation between the band 5 and the pressing cuff 52 provided on the inner circumferential surface of the curler 51 is not generated. Accordingly, the pressing cuff 52 and the sensing cuff 54 can be stably pressed toward the wrist, and thus blood pressure measurement accuracy can be stabilized and deterioration of the air bags 71 and 81 can be prevented.

In addition, the band 5 is provided on the outer circumferential surface of the curler 51, and thus the sag or the wrinkle is not generated in the band 5, and the high appearance can be obtained.

The curler 51 is formed to be longer than the length of the pressing cuff 52 in the longitudinal direction, and the band 5 is integrally attached to the outer circumferential surface of the curler 51 and extends from the end portion of the second curved portion 63 of the curler 51. Thus, the band 5 fastens the curler 51 to the wrist 300 by the pair of hook-and-loop fasteners 5a, and thus the curler 51 is elastically deformed to follow the shape of the wrist 300, so that the curler 51 can be fixed.

The curler 51 is made of the low-hardness material such as TPU, and the band 5 is made of a material that is less likely to stretch than the curler 51. Accordingly, when the pressing cuff 52 and the sensing cuff 54 are pressed toward the wrist 300 by the band 5 and fastened by the hook-and-loop fastener 5a, the curler 51 can be bent and deformed to follow the wrist 300. In addition, the curler 51 is configured such that an outer circumferential surface is covered with the band 5 in which the cloth material is made of the material that is less likely to stretch than the curler 51. Accordingly, when the pressing cuff 52 and the sensing cuff 54 are inflated, the curler 51 can be suppressed from being deformed outward in the radial direction. Thus, the curler 51 is easily elastically deformed to follow the shape of wrist 300, and can efficiently press the sensing cuff 54 toward the wrist 300 during the blood pressure measurement.

In addition, the curler 51 is formed by inserting the high-hardness material having hardness higher than that of the low-hardness material that is TPU and forming the curler 51 into the fixed portion 61 to be fixed to the device body 3. Thus, the intensity of the fixed portion 61 attached to the device body 3 can be improved, and thus the curler 51 can be prevented from being damaged even when the device body 3 is fixed by a screw or the like.

In addition, the device body 3 includes the loop portion 31a in the housing 11, and the band 5 is folded back at the loop portion 31a and then fixed by the pair of hook-and-loop fasteners 5a. Thus, the band 5 makes it easy to fasten the curler 51 and can bring the pressing cuff 52 and the sensing cuff 54 into close contact with the wrist 300 stably.

The first end of both ends of the curler 51 overlaps the second end of the both ends when the blood pressure measurement device is attached to the living body having a shortest length in a circumferential direction of at least the wrist 300 to which attachment is expected. so that the sensing cuff 54 can be brought into contact with the region where the arteries 311 and 312 are present even when the curler 51 is attached to any of the wrists 300 having different circumferences. That is, the blood pressure measurement device 1 prevents the both ends of the curler 51 from coming into contact with each other in the circumferential direction to generate a gap between the wrist 300 and the sensing cuff 54, and the pressing cuff 52 and the sensing cuff 54 can be brought into close contact with the wrist 300.

The first curved portion 62, which is the first end of the curler 51, has a smaller radius of curvature than the second curved portion 63 overlapping the first curved portion 62. Accordingly, the second curved portion 63 of the curler 51 is located outward in the radial direction than the first curved portion 62 before the band 5 is fastened, and thus the second curved portion 63 can be disposed on the outer side of the first curved portion 62 when fastened by the band 5. Further, when the curler 51 is fastened by the band 5, the second curved portion 63 can be wound up with a trajectory of a movement of the second curved portion 63 such that the second curved portion 63 covers the first curved portion 62, so that the second curved portion 63 can easily overlap the first curved portion 62. As described above, in the blood pressure measurement device 1, when the curler 51 is fastened by the band 5, the end portions easily overlap each other and the curler 51 is easily wound around the wrist 300.

Further, the blood pressure measurement device 1 is configured such that the band 5 is integrally attached to the outer circumferential surface of the groove 51a of the curler 51, and thus edges of the band 5 in a width direction face the pair of walls 51b of the curler 51. Accordingly, the edges of the band 5 in the width direction are covered by the pair of walls 51b, and thus the band 5 can be prevented from being peeled off by an external force from the outer circumferential surface of the curler 51.

The band 5 and the curler 51 are integrally formed, and thus the blood pressure measurement device 1 configured as described above has a good appearance, and can prevent the positional deviations between the band 5 and the curler 51 and between the band 5 and the air bags 71 and 81, and thus highly accurate blood pressure measurement can be performed.

Note that the present invention is not limited to the embodiments described above. For example, in the example described above, the configuration is described, in which the band 5 is fixed to the outer circumferential surface of the curler 51. However, the band 5 may be configured to include an unfixed portion or to include a notch in a direction orthogonal to the longitudinal direction in a portion of the curler 51, for example, a portion that is not exposed to the outside, for example, a portion present in the fixed portion 61. The band 5 is fixed to the curler 51 in a state of being formed in the band-like shape extending in one direction, and then the curler 51 is formed in a curved shape so as to follow the shape of the wrist 300. At this time, a difference between the inner and outer circumferences is generated in the curler 51 due to the thickness, and at this time, loosening may occur in the band 5 fixed to the curler 51. However, a part of the band 5 is not fixed to the curler 51 or includes a notch, even when a wrinkle or the like is generated in the portion due to the difference between the inner and outer circumferences of the band 5, the wrinkle is generated at a portion covered with the housing 11, and thus the wrinkle is not exposed to the outside and the appearance can be prevented from being impaired.

In addition, for example, the configuration is described, in which in the blood pressure measurement device 1, one of the first curved portion 62 which is the first end side of the curler 51 and the second curved portion 63 which is the second end side overlaps the other. Here, the curler 51 may be configured to include a guide portion 55 that guides one of the first curved portion 62 and the second curved portion 63 so as to move to an outer side when the one overlaps the other.

For example, when the second curved portion 63 moves to the outer side of the first curved portion 62 and the pressing cuff 52 and the sensing cuff 64 come into contact with the end portion of the first curved portion 62, the movement of the second curved portion 63 may be restricted. Thus, when the curler 51 includes the guide portion 55 that guides the second curved portion 63 to move to the outer side of the first curved portion 62, the second curved portion 63 rides on the end portion of the first curved portion 62 and easily overlaps the first curved portion 62.

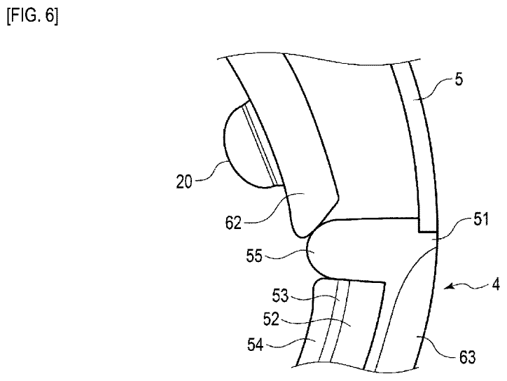

For example, as illustrated in FIG. 6, such a guide portion 55 is a protrusion that protrudes inward in the radial direction at the second curved portion 63. For example, the guide portion 55 is formed to be inclined with respect to a direction in which the guide portion 55 comes into contact with the end portion of the first curved portion 62 or to be a curved shape in which a tangential line is inclined. When such a guide portion 55 comes into contact with the first curved portion 62, and the distal end of the first curved portion 62 and the inclined surface come into contact with each other, and thus the guide portion 55 moves the second curved portion 63 in a direction intersecting a direction in which the guide portion 55 comes into contact with the first curved portion 62, that is, outward in the radial direction.

In addition, such a guide portion 55 is preferably formed higher than the sum of the thicknesses of the pressing cuff 52, the back plate 53, and the sensing cuff 54. That is, the distal end of the guide portion 55 is preferably located inward in the radial direction from the inner circumferential surface of the curler 51 than the sensing cuff 54. Accordingly, the second curved portion 63 is located outward than the first curved portion 62, and the second curved portion 63 can overlap the first curved portion 62. In addition, the guide portion 55 can prevent the first curved portion 62 from interfering with the pressing cuff 52, the back plate 53, and the sensing cuff 54. As described above, in the blood pressure measurement device 1, the second curved portion 63 can more reliably overlap the first curved portion 62 by providing the guide portion 55.

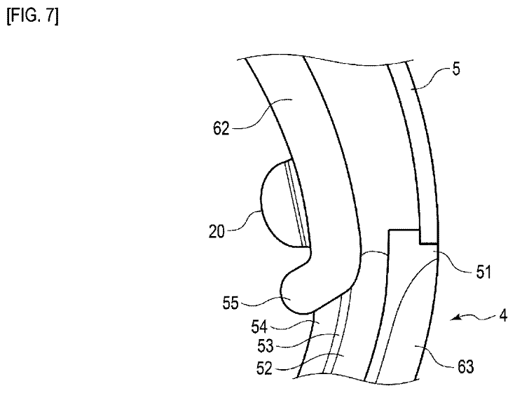

The guide portion 55 is not limited to the example illustrated in FIG. 6. For example, as illustrated in FIG. 7, the guide portion 55 may be a protrusion that is formed at the distal end of the first curved portion 62 and includes an inclined surface that is inclined with respect to a direction in which the first curved portion 62 and the second curved portion 63 come into contact with each other.

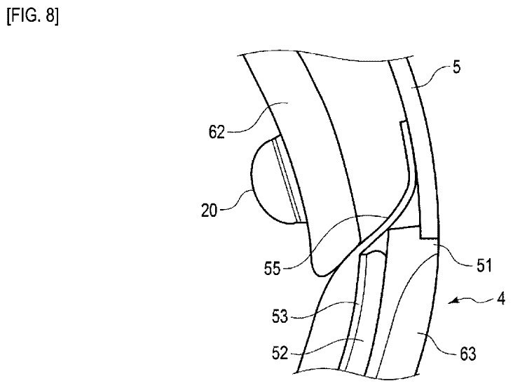

Further, as illustrated in FIG. 8, the guide portion 55 may be configured such that, for example, a sheet member is configured to make the air bag 81 of the sensing cuff 54 that is the outermost and the band 5 continuous with each other, and the end portions of the back plate 53, the pressing cuff 52, and the curler 51 are covered with this sheet member. For example, the guide portion 55 is formed by forming a part of the sheet member forming the air bag 81 in a shape longer than a shape of the air bag 81 and integrally attaching the sheet member to the band 5. Alternatively, for example, the guide portion 55 is formed by integrally attaching a rectangular sheet member separate from the air bag 81 to the outer surface of the air bag 81 and the band 5. With this configuration, the guide portion 55 forms an inclined surface that is inclined with respect to the direction in which the first curved portion 62 and the second curved portion 63 come into contact with each other, and guides the movement of the second curved portion 63.

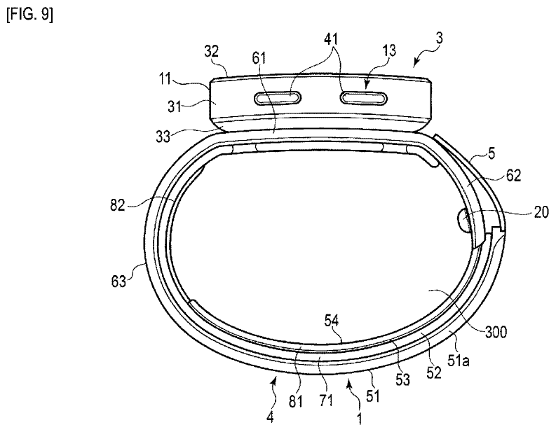

In the example described above, the configuration is described, in which in the blood pressure measurement device 1, the loop portion 31a is provided in the housing 11 and the band 5 is looped in the loop portion 31a. However, the configuration is not limited thereto. For example, as illustrated in FIG. 9, the blood pressure measurement device 1 may have a configuration in which the loop portion 31a is not included, and the hook-and-loop fastener 5a of the band 5 provided in the first curved portion 62 is joined to the hook-and-loop fastener 5a of the band 5 extending from the second curved portion 63.

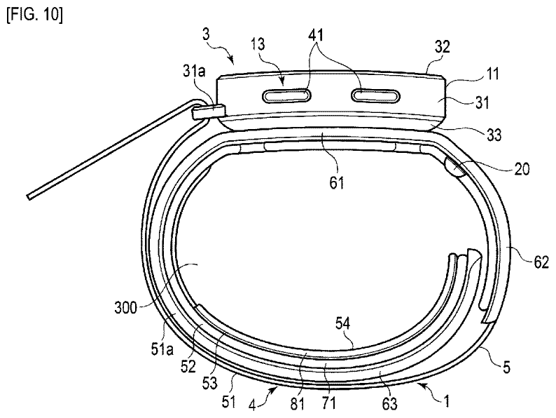

In the example described above, the configuration is described, in which in the blood pressure measurement device 1, the second curved portion 63 overlaps the outer side of the first curved portion 62. However, the configuration is not limited thereto. For example, as illustrated in FIG. 10, the first curved portion 62 may be configured to overlap the outer side of the second curved portion 63. In such a configuration, the band 5 may extend from the end portion of the first curved portion 62, and the loop portion 31a may be provided on the outer surface of the outer case 31 of the housing 11 on the side from which the second curved portion 63 extends.

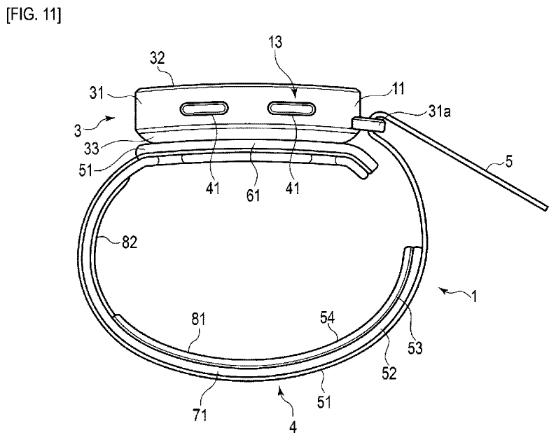

In the example described above, the configuration is described, in which the curler 51 has a shape longer than the pressing cuff 52, and the pressing cuff 52 is integrally provided on the inner circumferential surface of the curler 51. However, the configuration is not limited thereto. For example, as illustrated in FIG. 11, the curler 51 may have only the fixed portion 61 attached to the housing 11, and the pressing cuff 52 may be fixed to the inner circumferential surface of band 5 integrally attached to the fixed portion 61. In the case of such a configuration, the band 5 may include a layer made of, for example, TPU as a thermoplastic elastomer on the inner surface of the cloth material, and the band 5 and the pressing cuff 52 may be integrally fixed to each other by thermal welding.

That is, the present invention is not limited to the above-described embodiment, and various modifications can be made in an implementation stage without departing from the gist thereof. Furthermore, each of the embodiments may be carried out as appropriate in a combination as much as possible, and combined effects can be obtained in such a case. Furthermore, the inventions at various stages are included in the above-described embodiment, and the various inventions can be extracted in accordance with appropriate combinations in the plurality of disclosed constituent elements. Note that the present invention is not limited to the above-described embodiments, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### REFERENCE NUMERALS LIST

1 Blood pressure measurement device
3 Device body
4 Cuff structure
5 Band
5a Hook-and-loop fastener
11 Housing
12 Display unit
13 Operation unit
14 Pump
15 Acceleration sensor
16 Valve
17 Pressure sensor
18 Battery
19 Communication unit
20 Biometric sensor
21 Charging circuit unit
22 Memory
23 Processor
24 Fluid circuit
25 Substrate
26 Pogo pin
31 Outer case
31a Loop portion
32 Windshield
33 Back lid
41 Button
43 Touch panel
51 Curler
51a Groove
51b Wall
52 Pressing cuff
53 Back plate
54 Sensing cuff
55 Guide portion
61 Fixed portion
62 First curved portion
63 Second curved portion
64 Sensing cuff
71 Air bag
73 Connecting portion
81 Air bag
82 Flow path body
83 Connecting portion
100 Power transmission device
101 Power source
102 Power transmission unit
103 Antenna unit
211 Antenna unit
212 Power receiving unit
213 Charging unit
300 Wrist
311 Radial artery
312 Ulnar artery

1. A blood pressure measurement device comprising:
a device body;
a curler fixed to the device body;
a fluid bag fixed to an inner surface of the curler and inflated by a fluid; and
a band provided integrally with an outer surface of the curler or the outer surface of the curler and an outer surface of the fluid bag and including a hook-and-loop fastener on an outer surface of the band, wherein
a first end of both ends of the curler overlaps a second end of the both ends when the blood pressure measurement device is attached to the living body having a shortest length in a circumferential direction of at least the living body to which attachment is expected, and
the first end of the curler has a smaller radius of curvature than the second end overlapping the first end of the curler.

2. The blood pressure measurement device according to claim 1, wherein
the curler is formed longer than a length of the fluid bag in a longitudinal direction and is formed to be elastically deformable to follow a shape of an attachment site of a living body in a circumferential direction, and
the band is provided on an outer circumferential surface of the curler and extends from a first end of the curler.

3. The blood pressure measurement device according to claim 2, wherein
the curler is made of a low-hardness material and includes a fixed portion fixed to the device body and into which a high-hardness material having a hardness higher than a hardness of the low-hardness material is inserted, and
the band is made of a material that is less likely to stretch than the curler.

4. The blood pressure measurement device according to claim 1, wherein
the device body includes a loop portion configured to fold back the band.

5. The blood pressure measurement device according to claim 1, further comprising:
a guide portion configured to guide the second end overlapping the first end of the curler to an outer side of the first end.

6. The blood pressure measurement device according to claim 5, wherein
the guide portion is a protrusion provided at the first end or the second end of the curler and configured to move the second end to an outer side.

7. The blood pressure measurement device according to claim 5, wherein
the guide portion is a sheet member provided on an inner surface of the band, configured to cover an end portion of the fluid bag and extending from the curler.

8. The blood pressure measurement device according to claim 1, wherein
the curler is formed of a fixed portion made of a low-hardness material and a high-hardness material inserted into the low-hardness material and having a hardness higher than a hardness of the low-hardness material, the fixed portion being fixed to the device body, and
the outer surface of the fluid bag is integrally fixed to the fixed portion and the band.

9. The blood pressure measurement device according to claim 1, further comprising:
a sensor provided on an inner surface of a first end portion of the curler and separated from the fluid bag in a longitudinal direction of the curler and configured to detect information on a living body.
